(19) United States
(12) Patent Application Publication
(10) Pub. No.: US 2014/0039401 A1
(43) Pub. Date: Feb. 6, 2014

(54) SECUREMENT DEVICE

(71) Applicants: Marshall Kerr, Carlsbad, CA (US); Michael J. Vaillancourt, Chester, NJ (US); Marcia A. Ryder, Carlsbad, CA (US); Donn K. Harms, Del Mar, CA (US)

(72) Inventors: Marshall Kerr, Carlsbad, CA (US); Michael J. Vaillancourt, Chester, NJ (US); Marcia A. Ryder, Carlsbad, CA (US); Donn K. Harms, Del Mar, CA (US)

(21) Appl. No.: 13/956,311

(22) Filed: Jul. 31, 2013

Related U.S. Application Data
(60) Provisional application No. 61/677,972, filed on Jul. 31, 2012.

(51) Int. Cl. A61M 25/02

(52) U.S. Cl. CPC A61M 25/02
USPC 604/180

(57) ABSTRACT

A securement device is provided for engaging a catheter in an engaged position on a patient's skin. The device is formed of a flexible base having two half portions foldable to adhere to a sandwiched engagement of the catheter along a pathway. A tearaway portion in one half portion when removed forms an exit for the catheter to allow for changing of the securement device. Pads are provided in positions to communicate anti-pathogenic agents to the skin at the insertion site.

This application claims priority to U.S. Provisional Patent Application Ser. No. 61/677,972 filed on Jul. 31, 2012, and is incorporated herein in its entirety by this reference thereto.

### BACKGROUND OF THE INVENTION

1. Field of the Invention

The present invention relates to medical devices. More particularly, the disclosed device and method of employment, relates to a securement device configured for securing a catheter like device at an insertion site and concurrently providing a dressing to the site. In addition to securement of the catheter, the device herein provides means to communicate one or a combination of additional or supplemental anti-pathogenic agents or anti-bacterial agents, and/or phototherapy communication means to the insertion site and surrounding area, without the need for uncovering the site or surrounding area. In case of dressing disruption where the securement device requires replacement, or removal, patient comfort is enhanced by the ease of removal enabled by a tear off frangible portion of the device. Such allows for an easy removal of the device from engagement with a catheter, or kit components and the insertion site.

2. Prior Art

Intravenous catheters are employed in the medical field when there is a need to communicate fluids such as blood, plasma, saline solutions, and other medical treatments into the bloodstream of a patient. Examples of intravenous catheters include peripherally inserted central catheter (PICC) and others employed for long term and short term vascular access. While the term catheter is generally employed herein for such components, such devices for intravenous communication can also include, but are not limited to, a needle, an introducer, a catheter, a cannula, a handle portion, and one or a plurality of connectors for engaging the catheter to a source of intravenous fluid.

Conventionally, an insertion is made at the site of an underlying vein on a patient's skin. During engagement, a needle and introducer are employed to guide a catheter through the insertion site on the patient's skin and conventionally to the underlying vein. The catheter is inserted into the vein to the desired depth and then is preferably securely constrained.

This securement is conventionally accomplished by strips of adhesive tape or large planar sections of flexible adhesive material which overlay and secure the catheter to the skin to prevent it from inadvertently shifting or being pulled out. The insertion site is preferably protected through the employment of an antibacterial/anti-pathogenic treatment thereon, and is most commonly covered with a gauze pad or the like in order to reduce the chance of infection.

However, such conventional securement methods of the catheter and insertion site are known to be quite ineffective with regard to infection prevention, irritation prevention, and prevention of a dismount, since many catheters will remain in a patient for long periods of time and are subjected to a plethora of pathogens and dismounting contacts.

The conventional dressings and methods to provide adequate securement of the catheter at the insertion site often need to be changed frequently to avoid irritation to the patient and to help alleviate infections. Such removal is often a task that is unpleasant for both patient and medical professional since conventional securements do not allow for an easy dismount. Further, conventional mounts have failed to provide adequate communication of anti-pathogenic treatments to the insertion site and prevention of pathogens entering the insertion site. This greatly increases the chance of bacteria and pathogens entering the insertion site and causing infection.

Infection is the most common complication of long-term vascular access. One major cause of such infection is dressing disruption where the dressing providing the securement becomes wet or soiled and must be removed and replaced. It is also conventionally thought that central venous lines which are used for long-term access are prone to become infected at least once, with the highest rates of infection being in the neonatal population and in patients with short-bowel syndrome, neutropenia, or other illnesses. Venous thrombosis is another frequent complication of long-term venous access which can be caused by the type, size, and location of the catheter, as well as any preexisting conditions. In general, large catheters with multiple lumina tend to disturb the surrounding blood flow, leading to thrombosis.

When infection occurs, attempts may be made to salvage the catheter through the administration of antibiotics through the catheter. However, the most common method of treatment is to remove the catheter, as removing the infected catheter will help eliminate the source of infectious pathogens. Other methods of infection treatment include providing broad-spectrum antibiotics. However, such may not be suitable for all patients.

In recent years medical insurance companies and particularly Medicare, has deemed hospital-acquired infections as a medical problem that will preclude payment to the provider. As one can imagine, preventing catheter-related infection is a high priority for all healthcare workers. This can include strict hand-washing protocols, better handling of the catheter, and care of the insertion site via the securement and application of anti-pathogenic treatments.

As such, prior art has shown many attempts to provide improved dressing and securement devices for intravenous catheters intended to protect and prevent infection at the insertion site.

U.S. Pat. No. 3,918,446 to Buttaravoli teaches a securement device for intravenous catheters employed for holding a portion of the excess tubing while providing a cushion between the catheter-tubing assembly and the skin. However, the device is formed from many components which do not easily necessitate efficient application nor removal of the device.

U.S. Pat. No. 7,137,968B1 to Burrel et. al teaches a dressing device which is placed next to the skin, and a provided slit allows a bottom portion of the dressing to surround the catheter and insertion site. Further, the portion of the catheter protruding from the insertion site is in contact with an antimicrobial material of the dressing device. However, the configuration of the elongated slit does not necessitate secured engagement of the device surrounding the catheter and covering the insertion site. Further the antimicrobial material is not suited for prolonged communication of antimicrobial treatments to the site.

U.S. Pat. No. 4,059,105 to Cutruzzula et. al. teaches a cannula securing device comprising a unitary lamina having a narrow portion including an elongated opening employed to surround and expose the insertion site at which the cannula enters the patient, and a wide portion which when folded over the narrow portion, covers the opening and a portion of the tubing connected to the cannula and lying adjacent and essentially parallel to the length of the narrow portion. However, the minimal covering of the insertion site due to the opening employed to expose the insertion site greatly reduces the securement of the cannula to the patient and increases the risk of inadvertent catheter removable and exposure to bacteria.

Phototherapy bandage devices are also known in the medical field which employ specific wavelengths of light using lasers, light-emitting diodes (LED), fluorescent lamps, dichromic lamps or very bright, full-spectrum light. Medical treatment includes applications of light therapy for pain management, accelerated wound healing, hair growth, improvement in blood properties and blood circulation, and sinus-related diseases and disorders. However, conventional phototherapy bandage devices such as those found in U.S. Pat. Nos. 7,304,201 and 7,774,070, are not especially configured for employment as catheter dressing devices for communication of phototherapy while concurrently securing catheters and covering the insertion site.

These and similar devices fail to provide easy and effective engagement and adequate covering and securement of intravenous catheters to a patient. The longevity and effectiveness of the secured catheter is therefor greatly reduced, further reducing the patient comfort, and may inhibit the medical treatments they are receiving. Further these devices do not provide means for prolonged communication, and/or replenishment of antimicrobial treatments to the insertion site as is often necessary.

As such, there is a continuing unmet need for a catheter dressing and securement device providing improved engagement and support of the intravenous catheter at or near an insertion site. Such a device should communicate anti-pathogenic agents and concurrently provide a means for absorbing wound drainage at the insertion site.

In addition such a device should allow the user to easily position the device into the as-used position for proper positioning and securement of the device to the catheter and covering of the insertion site.

Such a device should advantageously provide a reservoir of an antimicrobial agent which can be communicated to the insertion site and surrounding area for prolonged periods of treatment. Such a reservoir should preferably be changeable or replenishable.

Further such a device should provide a means for easy removal of the device from the catheter and insertion site when the procedure ends, or in case of dressing disruption which requires a fast change of the dressing and securement to help avoid infection associated with such.

The forgoing examples of related art and limitations related therewith, are intended to be illustrative of the very different approach of different prior art devices and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The device herein disclosed and described provides a solution to the shortcomings in prior art of catheter securement devices configured to provide a dressing to a catheter insertion site. The device herein achieves such goals through the provision of an intravenous catheter securement device, and method, employing an easily registerable means for engaging an onboard reservoir of anti-pathogenic material in a secured positioning with a catheter and concurrently covering an insertion site. In addition to those named herein, the term anti-pathogenic material is intended to include any material employable to prevent or limit the growth of bacteria, viruses, and other pathogens which play havoc with the insertion sites of catheters. Further the device provides enhanced support and attachment means to secure the catheter in operable position on the patient's skin.

The device additionally includes a tear off or frangible portion which provides a means for easy removal of the device from the patient and the catheter, as needed. Through this releasable engagement with the catheter, allowing the catheter to be removed prior to the rest of the device herein, or the device to be disconnected prior to removal from the patient, there is provided a significant lessening of the pain and resulting stress on both patient and user practitioner. This means for disengagement from the catheter is especially useful in cases of dressing disruption where the dressing has become wet or soiled and needs replacement to prevent infection. During the replacement, the insertion site stays covered until the lower portion of the dressing herein is removed, and at virtually the same time, replaced. Further, because the catheter is disengaged from the securement device herein prior to removal of the adhesive dressing portion, contact with the catheter does not occur as is the case with the present art where the catheter may be translated, tugged upon, or juxtaposed during adhesive tape removal.

In accordance with a first preferred mode, the device is formed from conventional adhesive medical dressing or tape-like material, such as vinyl or polymeric material such as TEGADERM or any other suitable flexible adhesive bandage material. The disclosed device also includes at least one antimicrobial pad member providing a support and target for operative engagement with the catheter and a long term covering and communication of anti-pathogenic compounds impregnated to the pad, to the insertion site. This first pad member may be configured to communicate one or a plurality of anti-pathogenic agents at the insertion site and to the exterior of the catheter, and thus is preferably formed of material which may provide a reservoir for such anti-pathogenic compounds and concurrently provides a means for absorbing wound drainage at the insertion site.

This first pad member can be formed of one or a combination of materials which can serve as a dressing and a reservoir for anti-pathogenic agents, from a group dressing material including foam, hydro-colloid, polyurethane foam, polymeric pads, hydrofibers, medical gauze, biosynthetics, hydropolymers, alginates, or any other material suited for the intended purpose set forth in this disclosure. The anti-pathogenic agent can be one or a combination of anti-pathogenic and antibacterial agents from a group of such anti-pathogenic agents, including, manuka honey, silver nitrate, silver ions, aluminum ions, chlorhexidine gluconate (CHG), para chloro meta xylenol (PCMX), octenidine Di-hydrochloride (ODH), and other suitable material. Further, in addition to the first pad, the device can be configured for engagement of small auxiliary reservoirs of such agents, which may be communicated into the first pad, thereby allowing for change and/or replenishment thereof, without dismounting the device from the patient.

The device is preferably formed from two elongated halves or half sections which communicate at a central area between two side edges to form a hinge portion in the common central area. The hinge provides a means for folding one half section over the other half section, to achieve in a registered engagement with each other. So engaged, the two halved provide a sandwiched engagement with the catheter or venous line being communicated to the patient in such engagement. The hinge portion may engage the two portions via lateral side edges or bottom edges.

A first elongated half portion, defining the lower or skin adjacent portion of the device when folded and engaged in a used position on the skin, includes a top surface, bottom surface, and a proximal end wherein the first pad member is engaged, all defining a support area. The bottom surface includes an adhesive layer which is adapted to adhere to the user skin in a removably adhered engagement. In use and a used position, the first pad member is engaged over the insertion site and concurrently engages with and/or splayed around the catheter exiting the insertion site via a provided slot or gap in the pad member. This slot also provides a targeting means allowing the user to slide the device into position in the used position where the device is adhered to the skin.

While a rectangle or other shape will work, the support area in a favored mode, employs a triangulated means for engagement to the patient's skin, aiding the user to position device in the as used position in an engagement with the catheter, and to provide support for a covering which also surrounds over half of the insertion site or more.

While as noted a rectangular or round or other adhesive engagement will work, the triangulated means for adhesive engagement to the skin, is provided by at least two extending arm portions which are engaged traverse to, or configured in a substantially “Y”-shape which also maintains a secured mounted engagement of the device to the skin thorough the provision of resistive force to dismount from forces in many directions. Further, this “Y”-shaped portion, defines a skin gap between the two extending legs, which is uncovered and encourages the natural movement of patients' skin between the distal tips of the “Y” and therefor improved comfort to the patient.

Contact in this registered engagement with an inserted catheter and the first pad member is preferably provided by a slot extending from a terminating edge of the pad, a distance into a central area of the first pad, toward the distal end of the pad. There may be included a notched or gap portion at the terminating edge as a target to aid the user in guiding the catheter into the slot. Further, by using a slot or notched potion, the device when snugg up against the catheter using the slot as a target or guide, tends to cause a splay of the pad around a large portion of the catheter at the entry into the insertion site. This contact area communicates anti-pathogenic agents for contact with both the catheter, and the skin at the site. Impregnated with anti-pathogenic agents, the pad so splayed around the catheter and skin entry, significantly improves the ability of the device to prevent infection.

Further, in one preferred mode of the device the slot can be formed in a curvilinear manner, resembling a hook such that the pad member can achieve a hooked engagement with the catheter exiting the insertion site, or in a declining gap. This shaped first pad slot allows the pad member to completely surround and envelop the catheter in an engagement that aids in securely engaging the catheter thereon. This is of an advantage over prior art devices which provide linear slots which merely engage the catheter in an abutted engagement.

In accordance with at least one preferred mode of the device, the second half portion of the securement device herein has a top surface, a bottom surface, and proximal and distal ends. This second or top surface includes an adhesive section or layer positioned and formed such that when folded over, the adhesive layer engages pre-positioned non adhesive surface of the first component. This registered engagement provides for a sandwiched engagement of the catheter positioned therebetween and excellent securement to the patient. The device employs such adhesive layers which are covered by kraft or other paper adapted to easily release from the adhesive, until use. For ease of use of medical professionals who wear rubber gloves, pull tabs may be provided for the adhesive covers for easy removal prior to adherence.

The second portion additionally includes a second pad member which is configured and positioned thereon to engage over the support area of the first half portion when folded in half. The second pad member is preferably substantially circular to match the shape of the first pad member and sandwich the catheter, and contact the skin in the slot of the first pad. However, it may be of any other shape suitable for the intended purpose herein and could change if the first pad shape were to be changed to maintain the registered sandwiched engagement when the two halves are folded.

The second pad member may also be impregnated or otherwise formed to provide a reservoir of an anti-pathogenic agent to provide an additional means to communicate anti-pathogenic agents to the support area and insertion site for the term of engagement of the device to the patient. The bottom surface of the second half, when in the dismounted position, is preferably non-adhesive.

Particularly preferred, the second half portion includes perforations defining a frangible portion configured in a position to register and align with the section of the catheter in the sandwiched engagement. This frangible portion stays connected until deliberately torn and covers the remaining portion of catheter extending from the support area of the first half portion when in the used mode adhered to the skin.

After achieving the used position in an engagement adhered to the skin of the patient, and in a sandwiched engagement of the catheter with the first half portion and second half portion, if a new device is required due to contamination, or if the securement device is to be removed, the tear-a-way or frangible portion can be easily removed through peeling or pulling on a pull tab to remove it. Once removed, a gap is formed in the second half portion of the device aligned with the catheter, leaving the catheter exposed and where it can be disengaged without tugging on the adhered first half.

The provision of this frangible portion is a significant advantage over prior art, since the catheter can be removed without the need to completely remove the device from the adhered engagement with the user's skin, which will commonly tug on the user's skin and potentially irritate and cause pain to the patient from the catheter inserted in the vein or artery. Alternatively, the securement device herein can also be removed and replaced without any tugging or force imparted to the catheter which remains engaged at the insertion site, thereby avoiding a painful and potentially injurious moment for the patient. There is preferably included a pull tab portion extending from the distal end of the tearaway portion of the second half portion, to aid the user in grasping and removing it.

In further preferred modes, the top surface of the second half portion may include a mounting means engaged at or near the proximal end of the portion which is configured to removably engaged mounting components such as secondary reservoirs of anti-pathogenic fluid. As such, when folded over, the mount and an operatively engaged secondary reservoir will be positioned in a fluid communication with the second pad member of the second portion at or near the support area of the first half portion, and will communicate the anti-pathogenic to the underlying pad member and the skin surface. This can be changed if needed to add more or to change the anti-pathogenic agent to one adapted to prevent a new infection all without removing the securement device or causing patient pain and irritation.

In further preferred modes, the mount on the second half may also be configured to engage with a light projection means, such as an LED, which can be employed for photo therapeutic treatments of the insertion site, with the securement device in place. The LED or other light projection means may emit light in a frequency to kill germs and pathogens which is in the blue range or to encourage healing which is in the red range of the light spectrum. Again, without removing the securement device.

In yet another mode, the device is providable to the user in a kit. The kit may include a plurality of the bandage device and additional photo therapeutic treatment means. In one mode, photo therapeutic treatment light communication means can include an independently formed light projection component which is configured to engage the catheter over the pad member of the first half portion and can emit photo therapeutic light onto the insertion site. The kit may also include a catheter, introducer, and catheter fittings (such as a stat lock or luer lock) employing means for communicating photo therapeutic light to the insertion site. This may include direct communication through the device, or using a fiber optic communication from a remote light source and through the second half portion of the device.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Objects, features, and advantages of the invention will be brought out further in the following part of the specification, wherein detailed description is for the purpose of fully disclosing the invention without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features. It is intended that the embodiments and figures disclosed herein are to be considered illustrative rather than limiting. In the drawings:

FIG. 1 shows an overhead view of a particularly preferred mode of the catheter securement device.

FIG. 2 shows a bottom view opposite the view of the device of FIG. 1.

FIG. 3 shows a side view of the device if FIG. 1.

FIG. 4 shows a side view of another mode of the device employing removable backing layers.

FIG. 5 depicts a view of a particularly preferred phototherapy component employable in combination with the used mode of the device, to communicate light therapy to the insertion side or site being covered by the pads.

FIG. 6 shows a side view of the optional yet preferred mount, and mating mounting component where the mounting component can be one of a anti-pathogenic agent reservoir, or phototherapy light communication means.

FIG. 7 shows a preferred catheter component configured to additionally communicate photo therapeutic light, along the length of catheter tubing in a fiber optic fashion.

FIG. 8 shows a view of a first step in the procedure of engaging the device to a patient having an intravenous catheter inserted into their arm similar to that of FIG. 24.

FIG. 9 shows a view of a next step in the procedure of engaging the device to a patient to sandwich the catheter therebetween and whereafter optionally the phototherapy component of FIG. 5 may be engaged.

FIG. 10 shows a detailed view of the surrounding engagement of the first paid and the catheter where the device is adhered to the patient in the used position.

FIG. 11 depicts the folding step in the procedure of engaging the device to a patient and to secure the catheter, showing the device in the used mode with secured catheter extending.

FIG. 12 shows a view the step of removing the catheter from the patient, employing the novel tear-away portion to form a release of the catheter without dismounting the device from the skin.

FIG. 12a shows a collar style mounting engagement adapted for a sliding of a mounting component therein, to provide communication of light therapy through the pad or pads or to communicate more or different anti pathogenic agents to the second pad member.

FIG. 13 depicts a view of the device with the tear-away portion completely removed forming a removal passage and exposing the catheter.

FIG. 14 shows a preferred layered mode of the antimicrobial pad member having a reservoir portion and skin contact layer.

FIG. 15 shows a view of another particularly preferred mode of the invention wherein the two portions forming the device are vertically disposed about a foldable hinge formed in a central portion.

FIG. 16 shows the mode of the device of FIG. 15 in the folded, or as used position and achieving the sandwiched engagement.

FIG. 17 depicts a particularly preferred mode of the device providable to the user as a kit comprising one or a plurality of the securement device herein for engagement to patient's to secure catheters, phototherapy component, mount and mounting component, and a catheter.

FIG. 18 depicts another enlarged overhead view of the securement device showing the two halve portions engaged at a central portion defining a hinge and having paper or film covering adhesive on the second half portion.

FIG. 19 depicts an overhead view of the device of FIG. 18, with film or paper removed and showing adhesive on the second half portion and frangible portion and surrounding the second pad member.

FIG. 20 depicts an overhead view of the opposite side of that shown in FIG. 18, and showing the pull tab portion of the paper or film covering the first half portion.

FIG. 21 depicts the device of FIG. 20 with the film or paper removed and adhesive covered area surrounding the first pad member.

FIG. 22 depicts the insertion site on the skin of a patient with catheter extending therefrom.

FIG. 23 depicts a first step of removing the covering from the adhesive on the contacting surface of the first half portion of the device.

FIG. 24 depicts the sliding engagement of the device as configured in FIG. 23, to place the slot surrounding the catheter extending from the insertion site.

FIG. 25 shows a next step in removing the covering from the second half portion by pulling on the extending pull tab to expose the underlying adhesive.

FIG. 26 depicts the device adhered to the patient's skin with the second adhesive covering removed from the adhesive which is shown, and ready for the step of folding the device in the middle area, and onto itself.

FIG. 27 shows the device of FIG. 26 secured in a registered engagement of the first pad adjacent the second pad, and the elongated frangible portion aligned with and contacting the catheter. Also shown is an enlargement of the registered overlapping engagement of the first and second pads.

FIG. 28 depicts the step of removing the frangible portion from the second half portion by pulling on the tab.

FIG. 29 shows the result of removal of the frangible portion and formation of an exit channel in the area formerly occupied by the frangible portion.

### DETAILED DESCRIPTION OF THE PREFERRED EMBODIMENTS OF THE INVENTION

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation.

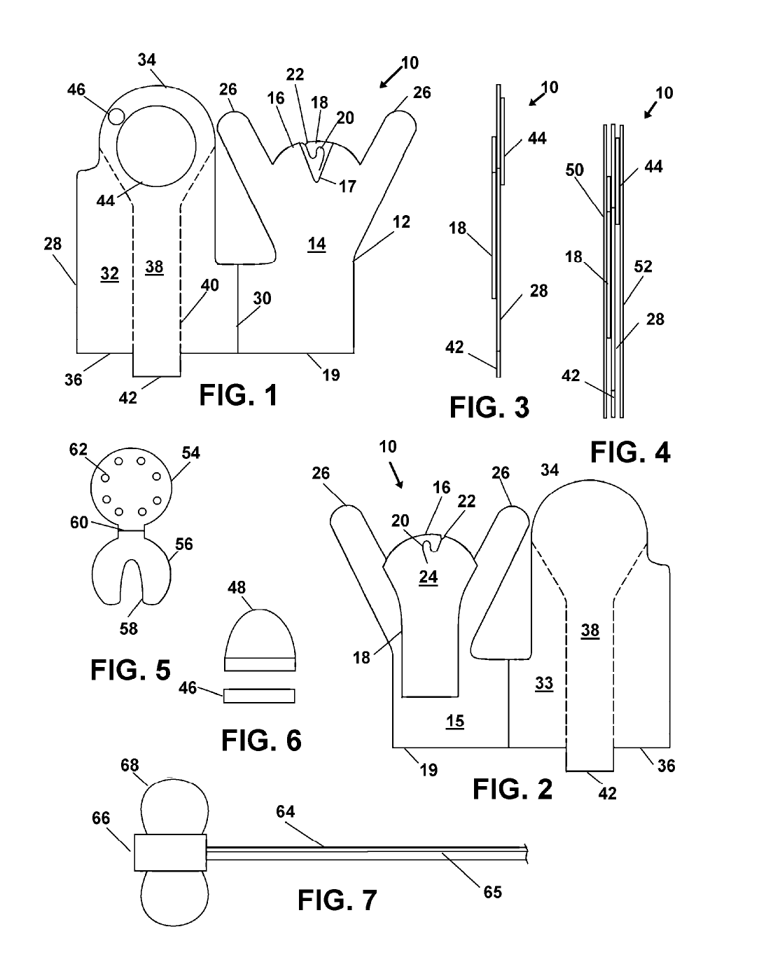

Now referring to drawings in FIGS. 1-29, wherein similar components are identified by like reference numerals, there is seen in FIG. 1, FIG. 2, and FIG. 3 views of a particularly preferred mode of the catheter securement device 10. The device 10 is preferably formed from a first half portion 12 and second half portion 28, which are held in operative engagement in a central area therebetween forming hinge portion 30 providing a means for folding one half over the other and into an as-used position, adhered to the skin of the patient, and in a sandwiched engagement with the catheter components such as in FIGS. 11 and 27.

Shown in the figures, the hinge 30 engages the lateral side edges of the respective half portions 12, 28 such that the folding procedure may be performed horizontally, (i.e right to left, or vis versa) to thereby achieves a registered engagement of the first half portion 12 with the second half portion 28 to position a sandwiched engagement of the catheter 64 underneath and aligned with a frangible portion 38. In accordance with a first preferred mode, the half portions 12, 28 may be formed from conventional adhesive medical securement material, such as polymeric or plastic material such as TAGADERM or any other suitable material.

The first half portion 12, defines a lower or skin-adjacent portion of the device 10 when in the folded mode (FIGS. 11 and 20 and 27). The first half portion 12 includes a top surface 14, bottom surface 15, distal end 19, and a proximal end 16 wherein a first pad member 24 is engaged. The first half portion 12 preferably includes a formed cut or notch 17 which is in a communication through the first pad member 24 to define an engagement and support area support area surrounding the insertion and catheter. In use the support area provides a protective covering that shields the insertion site 2000 and contacts the PICC line or other type of intravenous catheter 64 exiting the insertion site 2000.

The first pad member 18 is preferably configured to be impregnated with and/or communicate a reservoir of anti-pathogenic agents over time, at and around the insertion site 2000. Preferably it may also be layered to provide a contact section to provide a means for absorbing wound drainage at the insertion site 2000. To that end, the first pad member 24 can be one or a combination of pad forming materials from a group including foam, hydro-colloid, polyurethane foam, polymeric pads, hydrofibers, gauze, biosynthetics, hydropolymers, alginates, or any other material suited for the intended purpose set forth in this disclosure. It may be formed in layers or a laminate configuration to allow for the multiple actions noted.

The reservoir of anti-pathogenic agent can be impregnated into the material, or a layer thereof such as a gel, or otherwise formed within or in the pad member 18, or it may be also communicated to the first pad member 18 from a secondary reservoir engaged with one side surface of the device 10. The reservoir of communicated agent for all types of pathogens and bacteria and germs and the like anticipated in a hospital setting and/or at such insertions sites, can be one or a combination of anti-pathogenic agents from a group including, manuka honey, silver nitrate, silver ions, aluminum ions, chlorhexidine gluconate (CHG), para chloro meta xylenol (PCMX), octenidine Di-hydrochloride (ODH), and other suitable materials to the discerned pathogen causing the patient infection.

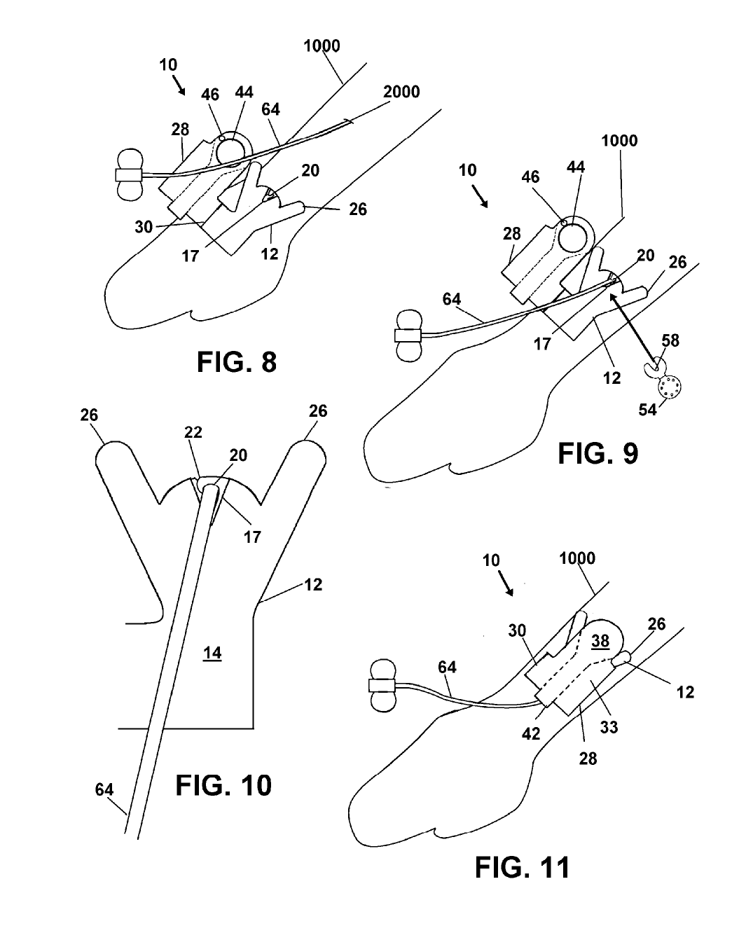

The contact or bottom surface 15 of the first half portion 12 includes an adhesive layer which provides means to removably engage the user skin when in the used mode. In use (FIGS. 8-13 and 27, and others) the first pad member 18 is positioned over and surrounding the insertion site 2000 and concurrently engages around the catheter 64 exiting the insertion site 2000 on a patient's skin such as on the arm 1000, or other location. As shown, the catheter 64 and first pad 28 engagement is enhanced via a formed slot either declining in cross-section or a shaped curvilinear slot 20 extending from a proximal terminating edge 16 of the pad 18 a distance toward the distal end 19. There may also be included a notch 22 or 17 at the terminating edge 16 of the slot 20 to aid the user in guiding the catheter 64 into the slot 20.

Slot 20 is preferably formed in one mode in a curvilinear manner resembling a hook as can be clearly seen in the figures. As such, the support area of the pad member 18 can achieve a hooked splayed engagement with and around the circumference or most thereof, of the catheter 64 (FIG. 10) exiting the insertion site 2000. This mode of the slot allows the first pad member 18 to completely surround and envelop the catheter 64 and insertion site, in an engagement that aids in securely engaging the catheter thereon and completely covering the insertion site 2000 to prevent communication of bacteria to the site 2000 for reducing infection.

The device 10 is configured to provide a means for a triangulated engagement of the device 10, with the skin 1000 and with the engaged catheter 64 by providing a multi angle resistive support to any dismount, from multiple opposing angles. This configuration provides means for aiding the user to position device 10 in the used position (FIGS. 9 and 11 and 27) in an aligned registered engagement with the catheter 64, and covering the insertion site 2000.

The legs and body of the device forming the triangulated engagement means provides multiple support adhered surfaces at converging vectors by employing at least two extending arm portions 26 which are configured traverse to the axis of the engaged catheter 64, in a substantially “Y”-shape. The “Y” shape as shown in the figures, with the catheter 64 aligned with the axis of the “Y”, additionally provides a means to maintain a secured mounted engagement of the device 10 to the skin of the patient. Further, the “Y”-shaped portion encourages the natural movement of patients skin between the distal tips of the “Y” and therefor improved comfort to the patient.

The second half portion 28 is formed of a mating or top surface 32, a bottom surface 33, and proximal 34 and distal 36 ends or edges. The mating or top surface 32 preferably includes an adhesive layer such that when folded over, will achieve an adhered engagement with the top surface 14 of the first half portion 12 and the catheter 64.

The second half portion 28 additionally can preferably include a second pad member 44 which is configured to roll or rotate to a registered engagement with, and communicate over, the first pad 24 and support area of the first half portion 12 when folded thereover. The second pad member 44 is shown substantially circular as to provide a substantial matching covering of the notch 17 and first pad 24 and support area. However, it is noted that the pad 44 can be any shape suitable for the intended purpose and may change to match a changed shape of the first pad 24.

The second pad member 44 may also preferably be impregnated with or otherwise formed to hold a time-dispensed reservoir of an anti-pathogenic agent to the insertion site and to the first pad member 24, to provide an additional means to recharge the first member and to communicate anti-pathogenic agents to the insertion site 2000 due to the proximity to the support area in the used mode, covering and contacting the first pad member 24.

FIG. 4 shows a particularly preferred mode wherein the device 10 employs one or a plurality of removable backing layers 50, 52, for removably covering the adhesive on the two half portions which is especially well configured for packaging and storing the device 10. The backing layers 50, 52 will cover the adhesive surfaces of the device 10, and the user will remove the prior to operative employment of the device 10.

The second or bottom surface 33 of the second half portion 28 is preferably non adhesive, as it will be the exposed surface when folded over in the used mode shown in FIGS. 11 and 27. To aid in the removal of the catheter 64 from the device 10, and patient comfort, there is included at least one perforation-defined, frangible, or tear-away portion 38, which with the two half portions in registered engagement, aligns with the axis of the length of catheter 64, extending from the insertion site 2000.

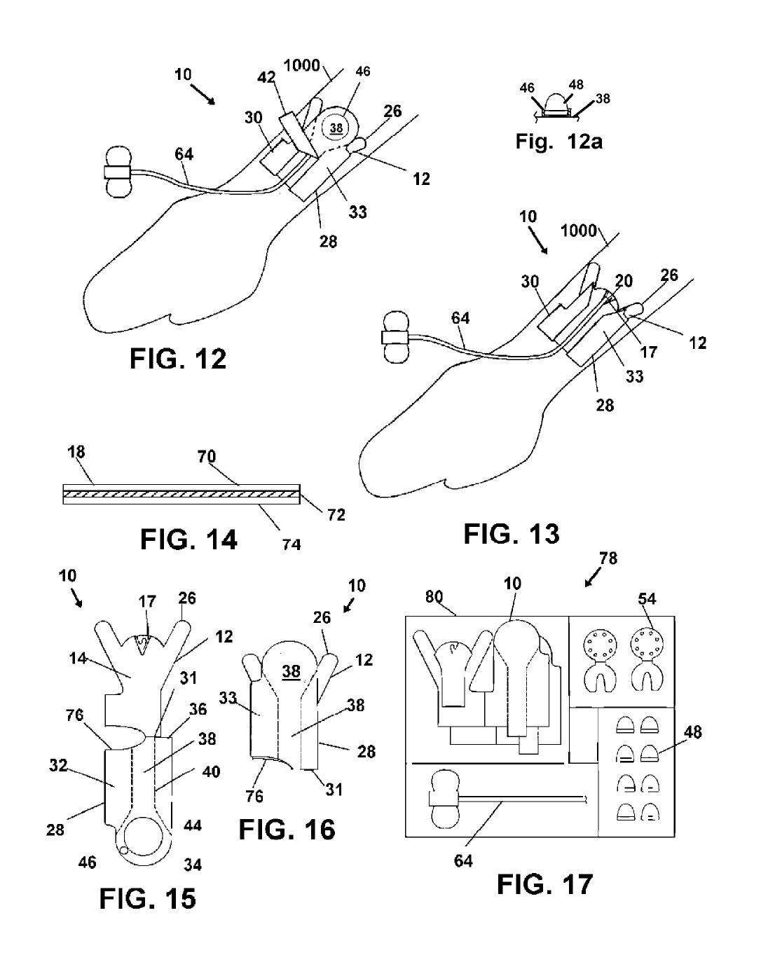

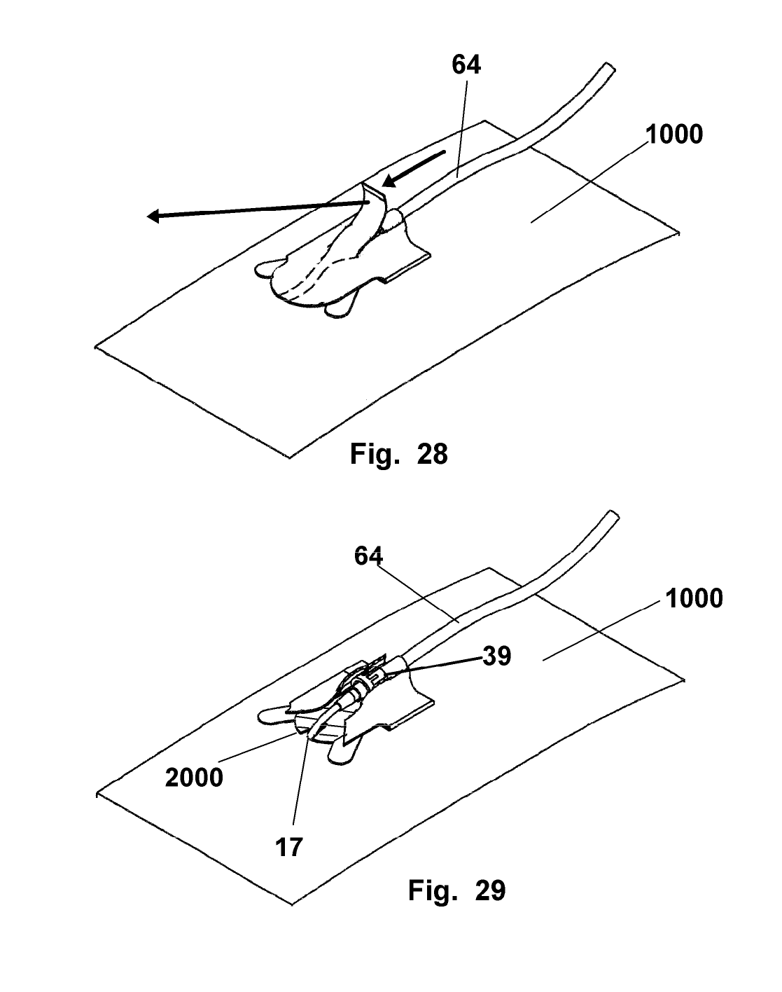

Shown in FIG. 12 and FIGS. 13 and 25 for instance, a tab 42 of the tear-a-way or frangible portion 38 defined by perforations extends past the edge of the central portion 30 of the body of the device 10, and may be easily gripped with gloved fingers and pulled away from the device 10, and remove the frangible portion 38 as shown in FIGS. 28-29. As such, the frangible portion 38 provides a means to expose a passage 39 and thereby provide a disengagement of the underlying catheter 64, through the wall forming the second half portion, so that the securement device 10 or the catheter 64 may be changed or removed without imparting force to the catheter 64 or the patients skin or insertion site, all of which can be painful to the patient.

This removable or frangible portion 28 being torn away easily, is a particular advantage over prior art since the catheter 64 can be removed without the need to completely remove the device 10 (i.e the first portion 12 and the remaining parts of the second portion 28) from the adhered engagement with the users skin, which will commonly tug on the users skin and potentially irritate the catheter 64 inserted in the vein. This affords an easy replacement of the device 10, or removal of the catheter 64 without first removing the first half portion from the skin of the patient.

In additional preferred modes, the exposed or top surface 32 of the second half portion 28, exposed when in the used position, may include a mount 46 engaged at the proximal end 34. The mount 46 is shown but can be moved to be preferably configured to removably engage one or a plurality of mounting auxiliary components 48, in operative engagement with the device 10 shown in FIG. 6. The mounting auxiliary components 48 may be one or a combination of such components from a group including time release, or time delayed, or other reservoirs or supplies of anti-pathogenic fluids or agents, or, a light projection means for providing phototherapy to the insertion site when in the folded over as used mode. Communication of such agents or light therapy would be through the wall surface of the second half portion or an aperture formed therein.

In the modes employing light projection means, a power source (such as a battery) and LED or other powered light emitter will be included as part of the light mounting component 48 or provided in a communication therewith from a battery or power source or engaged switch allowing for timed or pulsed communication of light to the insertion site and down the catheter if formed of polymeric material configured therefor, such as a catheter having a noticed exterior configured to capture transmitted light, and communicate such light along the path of the catheter wall through the insertion site and into the patient.

If a switched power supply is engaged, a microprocessor may be employed to pulse the communicated light and/or provide light therapy at timed intervals to the insertion site and surrounding area to aid in the killing of pathogens and tissue regeneration. Different wavelengths of light may be communicated to the insertion site by the employment of LED's or other light generation means, in operative engagement with a mounting component 48. For instance, light in the red wavelengths encourages cell regeneration while light in the blue wavelengths kills pathogens. In that fashion, using the employed switch or software running on a microprocessor, different wavelengths of light therapy can be communicated in a single session or multiple sessions as needed.

In the folded, as-used mode, in registered engagement of the two half portions, the mount 46 and an operatively engaged mounting component 48 is operatively positioned over or adjacent the support area of the first half portion 12 and through the close proximity can communicate the anti-pathogenic agent or light through a contact or communication with the underlying pad member 44 and/or through a communication through the bandage portion to the insertion site 2000.

In the case of light therapy, a bandage forming the second half portion, and parts of the first half portion, of light transmitting material will allow the insertion site to stay covered even with the addition of light therapy.

Further, a mounting component 48 with an additional supply of an alternative or the same antipathogenic agent also allows for supplemental treatment without uncovering the insertion site and risking contamination. The mount 46 engages the bandage using attachment means such as adhesives, and as shown in FIG. 12a the mount 46 is formed as a collar type, bayonet, or other frictional or mechanical mount for the mounting component 48. The mounting component 48 thus operatively engages thereto in a removable engagement to allow for replacement or substitution or additional antipathogenic therapies without uncovering the insertion site and inviting the perils which occur conventionally after a securement disruption.

FIG. 5 and FIG. 9, show a particularly preferred phototherapy mounting component 54, which is employable in combination with the device 10 in the as-used mode. The phototherapy mounting component 54 includes a catheter engagement portion 56 having a notch or recess 58 configured to engage around the exterior surface of the catheter 64. A light projection portion 62 is also provided and includes a plurality of phototherapy light-emitting components, such as LED's as noted above which may transmit light on one or a plurality of differing wavelengths.

As shown in FIG. 9, a means for light emitting phototherapy mounting component 54 can be optionally employed with the device 10 in the used mode by engaging a recess 58 around the exterior of the catheter 64, extending from the insertion site 2000, then folding the light projection portion 62 about a common hinge 60 such that the light emitters project over the support area and insertion site 2000 to provide light therapy thereto and/or down the wall of the catheter, which may be configured to receive light transmissions exterior to the insertion site, and to broadcast them at the insertion site and below the skin such as with notches in the exterior of the catheter 64 forming projecting or fresnel lens configuration portions of the wall of the catheter.

FIG. 7 shows a view of a particularly preferred catheter 64 employing means for phototherapy and one or a plurality of wavelengths as well. The catheter 64 as earlier noted, can be formed with fiber optic 65 portions to communicate light provided by the phototherapy component 54, into the insertion site 2000. In addition, the catheter fitting 66, conventionally called a luer lock or STATLOCK, can include planar wing portions 68 having light-receptive coatings, or configurations such as a prism, which capture and communicate the transmitted phototherapy light from the phototherapy component 54, to the fiber optics 65 of the catheter 64. The catheter 64 under the skin of the patient would have notches or prisms or fresnel lenses or the like formed in the wall thereof, to cause a communication of the light traveling through the wall axially, and light emissions below the skin surface as a means to kill pathogens. The light in all modes may be provided in one or a plurality of wavelengths, and may be pulsed, continuous, or at timed intervals. Light therapy in the 620-660 nm range may be employed for pain management, accelerated wound healing and cell growth, improvement in blood properties and blood circulation.

As noted, transmitted light in the blue range of (400-600 nm) may be employed to kill bacteria that are frequently found in infected wounds with 400-480 nm being particularly favored for this purpose. These ranges and others are anticipated for employment herein singularly or in combination, in continuous, timed, or pulsed modes, and combinations thereof.

FIG. 12a shows a collar style mount 46 adapted for a sliding of a mounting component 48 therein to provide light therapy or to communicate more or different anti pathogenic agents to the pad member 44 to enhance performance if resistance is noted to the pathogenic agent being used. A frictional engagement or a mechanical engagement can be provided by the collar style mount 46 with the mounting component 48 having light therapy or communicable pathogenic agents thereon. This allows for the addition of supplemental antipathogenic agents such as silver ions or aluminum ions to the pad member 44, or light therapy to the insertion site and surrounding areas with a phototherapy component 54, after engagement of the device without uncovering the insertion site. Keeping the securement bandage in place covering the insertion site, while allowing for supplemental medications and therapies such as light therapy to enhance healing or kill pathogens, avoids the pitfalls of securement disruption which currently are a major source of infection.

FIG. 14 shows cross sectional view of a preferred mode of the pad members 18, 44 of the device 10. In this mode the pads 18, 44 can be formed from a plurality of planar layers, for example a first layer of foam or padding 70, an intermediate layer of anti-pathogenic impregnated material 72, and a second layer of foam or padding 74 or fluid absorbing material to aid drainage. Various other combinations and pluralities of layers may also be employed and are anticipated. The layering of the pads 18, 44 can provide a means to regulate the rate of communication of the anti-pathogenic agent out of the pad 18, 44 and onto the insertion site 2000 and surrounding skin, and the rate can be of the designers choice. Any or all of the pads described herein may be employed on any mode or configuration of the device herein, and may be employed without, or in combination with the light therapy emitting components herein.

FIG. 15 and FIG. 16 show unfolded and folded positions of yet another mode of the securement device 10. In this mode the first and second portions 12, 28 are engaged about a hinge 31 disposed on the distal edges 19, 36 such that the folding procedure is done vertically (i.e top to bottom, or vis versa). There is additionally included a notched portion 76 which will allow the catheter 64 to communicate from the support area, through a sandwiched engagement between the first portion 12 and second portion 28, and out and away from the device 10.

FIG. 17 shows yet another preferred mode wherein the device 10 is providable to the user in a kit 78. The kit 78 may include a plurality of the bandage device 10, phototherapy mounting component 54, mounting component 48, and catheter 64. The kit 78 may also include an introducer and catheter fittings (such as a STATLOCK or luer lock) employing means for communicating photo therapeutic light to the insertion site 2000. The kit 78 may be providable in a sterilized package 80.

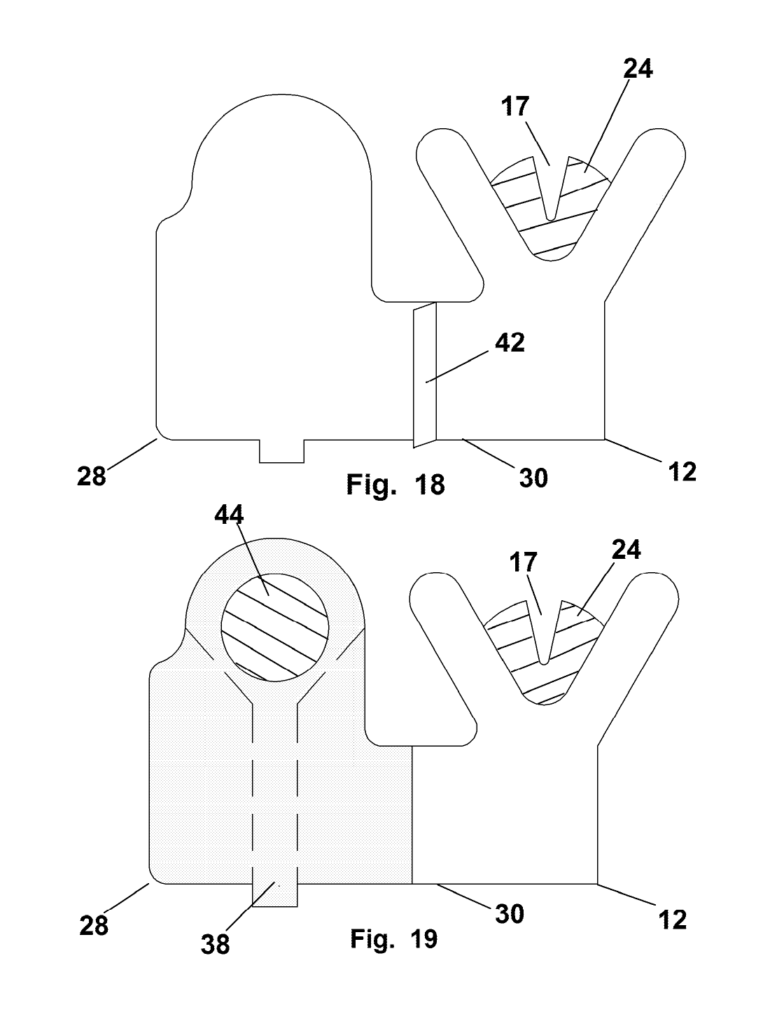

FIG. 18 depicts another enlarged overhead view of the securement device 10 showing the two halve portions 12 and 28 engaged at a central portion 30 defining a hinge and having paper or film covering adhesive on the second half portion which extends to a tab 42 for dismount.

FIG. 19 depicts an overhead view of the device of FIG. 18, with film or paper removed by pulling on the tab 42, and showing adhesive 43 on the second half portion 28 and frangible portion 38 and also surrounding the second pad member.

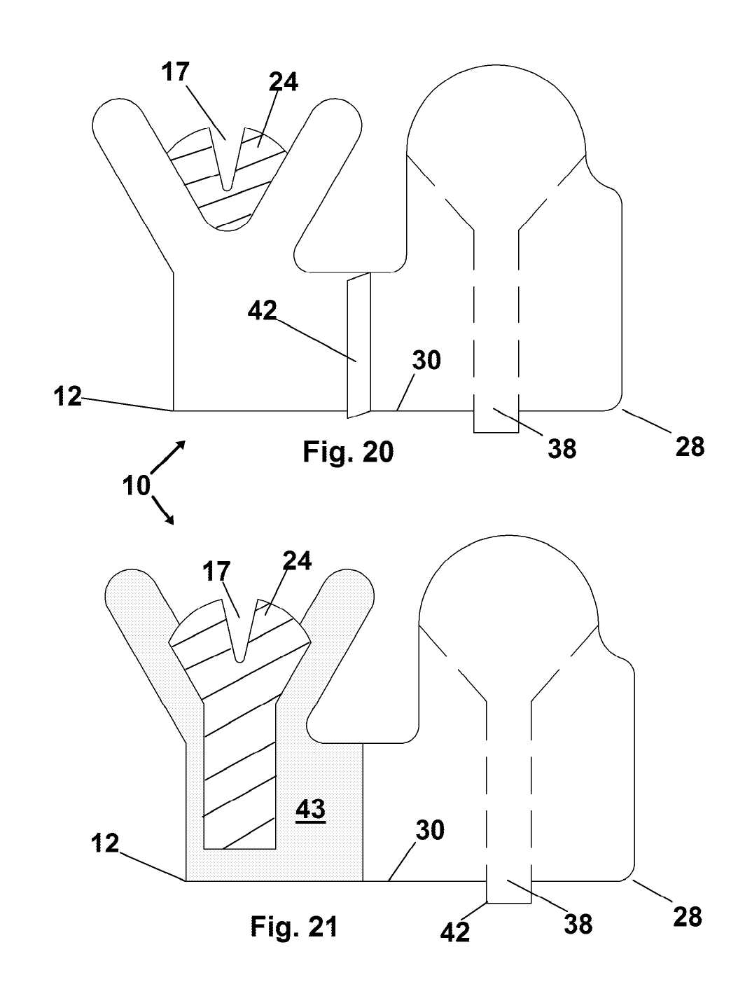

FIG. 20 depicts an overhead view of the opposite side of that shown in FIG. 18, and showing the pull tab 42 portion of the paper or film covering the adhesive on the first half portion 12.

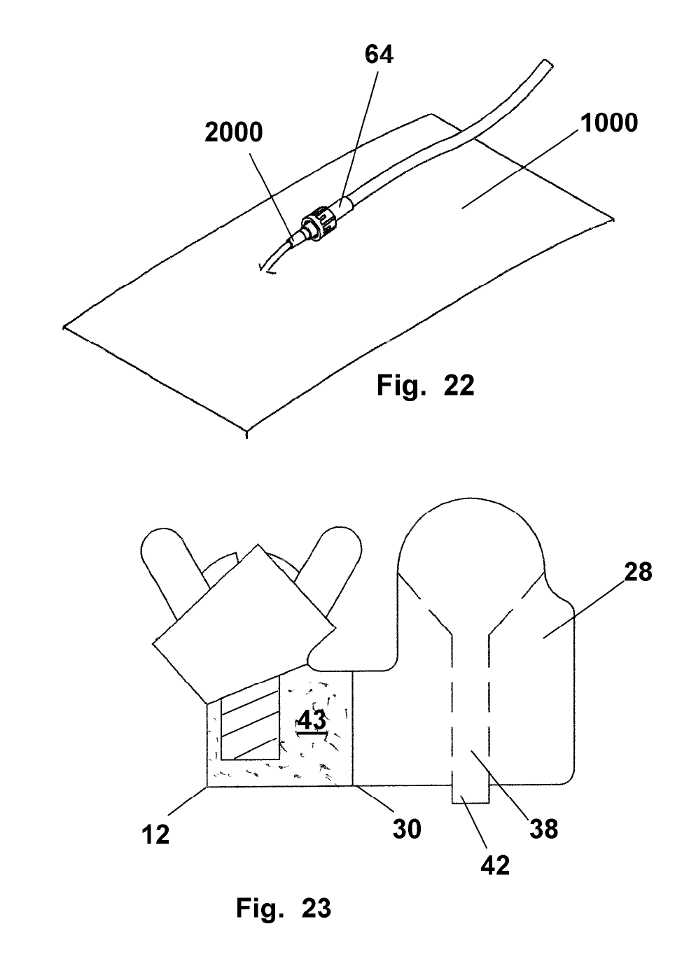

FIG. 21 depicts the device of FIG. 20 with the film or paper removed and adhesive 43 covered area surrounding the first pad member 24. FIG. 22 depicts the insertion site 2000 on the skin of the arm 1000 of a patient with catheter 64 extending therefrom and ready for engagement with the device 10.

FIG. 23 depicts a first step of removing the covering from the adhesive 43 on the contacting surface of the first half portion 12 of the device 10.

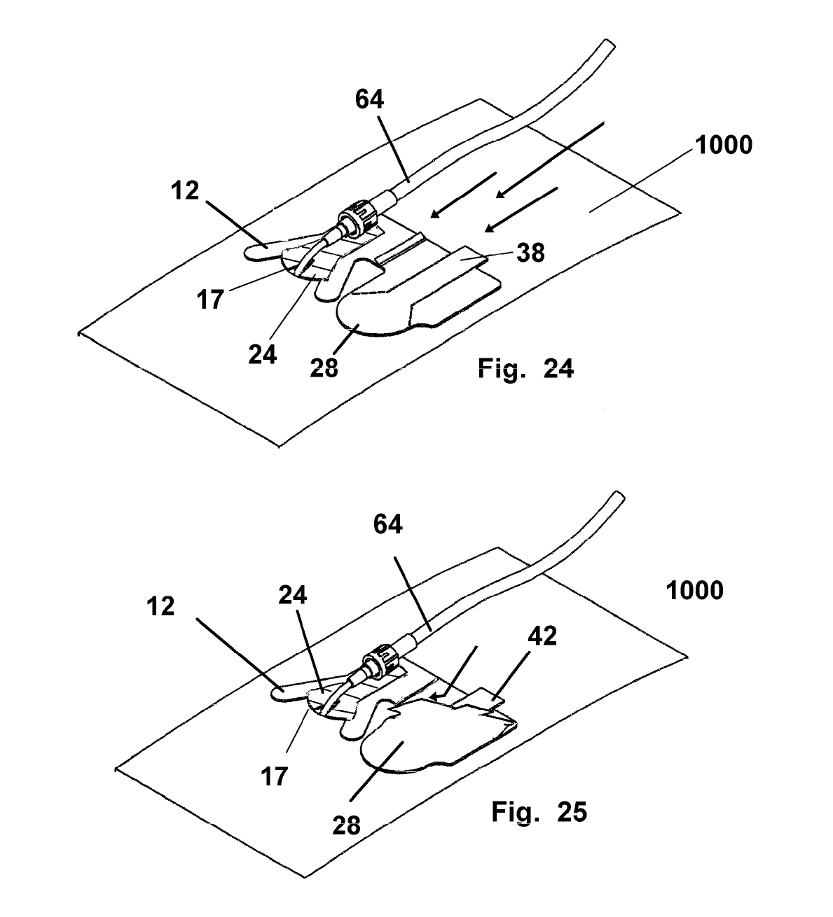

FIG. 24 depicts the sliding engagement of the device 10 as configured in FIG. 23, to place the slot formed in the first pad 24 to a position abutting and surrounding a portion of the circumference of the catheter 64 extending from the insertion site.

FIG. 25 shows a next step in removing the covering for the adhesive 43 on the second half portion 28 by pulling on the extending pull tab 42 to expose the underlying adhesive 43.

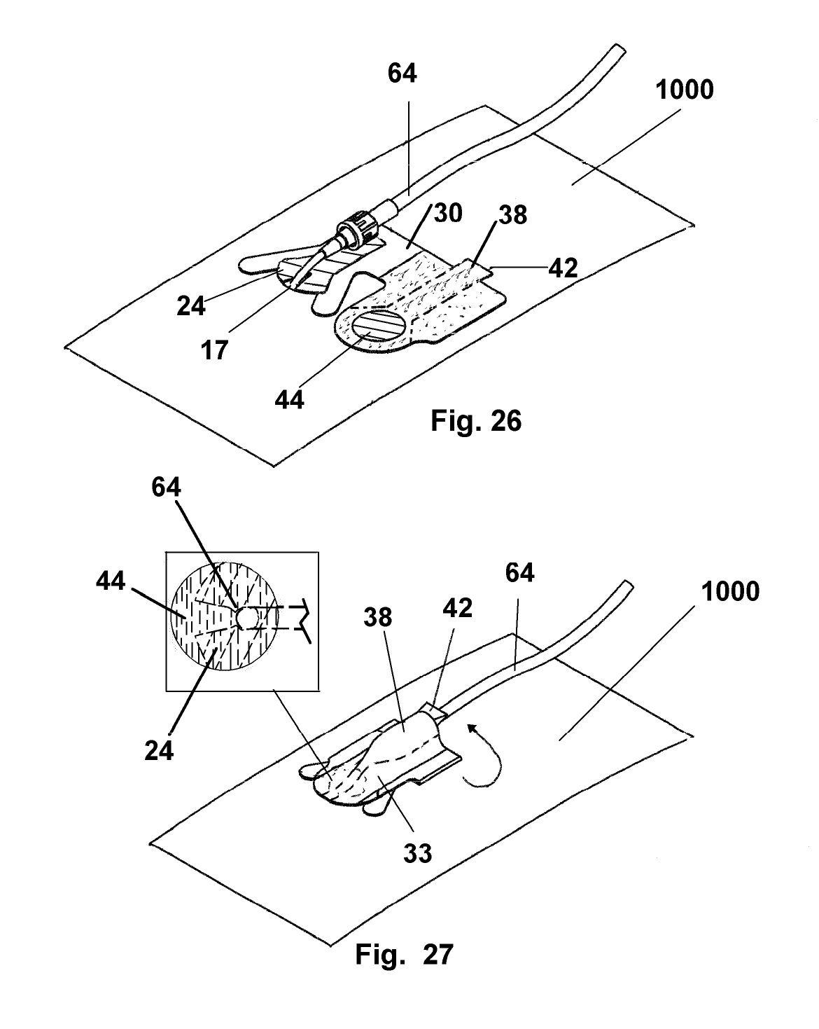

FIG. 26 depicts the device 10 adhered to the patient's skin of the arm 1000 with the second adhesive covering removed from the adhesive 43 which is shown, and ready for the step of folding the device 10 in the middle area 30, and onto itself.

FIG. 27 shows the device of FIG. 26 secured in a registered engagement of the first pad 24 adjacent the second pad 44, and the elongated frangible portion 38 aligned with and contacting the catheter 64. Also shown is an enlargement of the registered overlapping engagement of the first and second pads.

FIG. 28 depicts the step of removing the frangible portion 38 from the second half portion 28 to expose the exit passage 39 for the catheter 64 by pulling on the tab 42 to remove the tear away section defined by the frangible portion 38.

FIG. 29 shows the result of removal of the frangible portion 38 and formation of an exit passage 39 in the area formerly occupied by the frangible portion 38. As noted this allows the device 10 and/or the catheter 64 to be disengaged so that removal of either does not tug on the other and injure or cause pain to the patient.

The invention of the disclosed device herein has other applications, potentially, and one skilled in the art upon reading this disclosure and being educated with regard to this device and method could discover such. The disclosure and explanation of the features of the disclosed device does not limit the claims of this application, and other applications developed by those skilled in the art are considered to be included in this invention.

It is additionally noted and anticipated that although the device is shown in its most simple form, various components and aspects of the device may be differently shaped or slightly modified when forming the securement device herein. As such those skilled in the art will appreciate the descriptions and depictions set forth in this disclosure or merely meant to portray examples of preferred modes within the overall scope and intent of the invention, and are not to be considered limiting in any manner.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. A securement device for engaging a catheter in position on a patient's skin, comprising:
a flexible base having a first half portion and having a second half portion;
a central portion positioned between said first half portion and second half portion;
a first adhesive surface on a contact side of said first half portion, opposite a second side thereof, said first adhesive surface adapted for adhesive engagement to said patient's skin to position said base to a mounted position;
a second adhesive surface positioned upon said second half portion;
a first pad engaged with said first half portion at a proximal end;
an opening communicating into a first edge of said first pad adjacent said proximal end, to a central area of said first pad;
said opening adapted to abut a portion of an exterior surface of said catheter exiting an insertion site upon said skin of said patient, when said first half portion is positioned to said mounted position;
said first pad positioned upon said first half portion to place a contact side thereof, adjacent said insertion site when said first half portion is placed in said mounted position;
said central portion foldable to position said second adhesive surface upon said second half portion in an adhesive engagement upon said second side of said first half portion;
said engagement forming a sandwiched positioning of a portion of said catheter along a pathway running in-between said first half portion and said second half portion; and
said sandwiched positioning with said base in said mounted position providing a mount for said catheter to prevent translation thereof through said insertion site.

2. The securement device of claim 1 additionally comprising:
said first pad formed of material impregnated with a first reservoir of an anti-pathogenic agent; and
said first pad providing means for communicating said anti-pathogenic agent to said skin of said patient surrounding said insertion site.

3. The securement device of claim 1 additionally comprising:
a tear-away portion formed in said second half portion;
said tear-away portion positioned to parallel said pathway of said sandwiched engagement of said catheter;
removal of said tear-away portion forming an opening in said second half portion, along said pathway; and
said opening providing means for removal of said catheter from said sandwiched positioning along said pathway, and a subsequent movement of said base from said mounted position, without a disengagement of said second half portion from said adhesive engagement with said first half portion.

4. The securement device of claim 2 additionally comprising:
a tear-away portion formed in said second half portion;
said tear-away portion positioned to parallel said pathway of said sandwiched engagement of said catheter;
removal of said tear-away portion forming an opening in said second half portion, along said pathway; and
said opening providing means for removal of said catheter from said sandwiched positioning along said pathway, and a subsequent movement of said base from said mounted position, without a disengagement of said second half portion from said adhesive engagement with said first half portion.

5. The securement device of claim 1 additionally comprising:
a second pad engaged with said second half portion;
said second pad positioned upon said second half portion to form a sandwiched contact of a portion of said catheter exiting and adjacent said insertion site, between said first pad and said second pad;
said second pad formed of material impregnated with a second reservoir of an anti-pathogenic agent; and
said second pad in said sandwiched contact, providing means for communicating said anti-pathogenic agent, to said surface of said catheter and to any said skin of said patient surrounding exposed within said opening of said first pad.

6. The securement device of claim 2 additionally comprising:
a second pad engaged with said second half portion;
said second pad positioned upon said second half portion to form a sandwiched contact of a portion of said catheter exiting and adjacent said insertion site, between said first pad and said second pad;
said second pad formed of material impregnated with a second reservoir of an anti-pathogenic agent; and
said second pad in said sandwiched contact, providing means for communicating said anti-pathogenic to said surface of said catheter and to any said skin of said patient surrounding exposed within said opening of said first pad.

7. The securement device of claim 3 additionally comprising:
a second pad engaged with said second half portion;
said second pad positioned upon said second half portion to form a sandwiched contact of a portion of said catheter exiting and adjacent said insertion site, between said first pad and said second pad;
said second pad formed of material impregnated with a second reservoir of an anti-pathogenic agent; and
said second pad in said sandwiched contact, providing means for communicating said anti-pathogenic agent, to said surface of said catheter and to any said skin of said patient surrounding exposed within said opening of said first pad.

8. The securement device of claim 4 additionally comprising:
a second pad engaged with said second half portion;
said second pad positioned upon said second half portion to form a sandwiched contact of a portion of said catheter exiting and adjacent said insertion site, between said first pad and said second pad;
said second pad formed of material impregnated with a second reservoir of an anti-pathogenic agent; and
said second pad in said sandwiched contact, providing means for communicating said anti-pathogenic agent, to said surface of said catheter and to any said skin of said patient surrounding exposed within said opening of said first pad.

9. The securement device of claim 7 additionally comprising:
said opening formed as a declining gap; and
a wider portion of said gap providing a targeting means for a sliding of said gap around said catheter exiting said insertion site.

10. The securement device of claim 7 additionally comprising: said opening formed as a declining gap; and
a wider portion of said gap providing a targeting means for a sliding of said gap around said catheter exiting said insertion site.

11. The securement device of claim 7 additionally comprising:
a secondary mount formed on a side surface of said second half portion opposite said second pad and aligned or adjacent therewith; and
said secondary mount configured for engagement of a photo therapeutic light.

12. The securement device of claim 8 additionally comprising:
a secondary mount formed on a side surface of said second half portion opposite said second pad and aligned or adjacent therewith; and
said secondary mount configured for engagement of a photo therapeutic light.

13. The securement device of claim 7 additionally comprising:
a secondary mount formed on a side surface of said second half portion opposite said second pad and aligned or adjacent therewith; and
said secondary mount configured for engagement of a supplemental reservoir of a said anti pathogenic agent.

14. The securement device of claim 8 additionally comprising:
a secondary mount formed on a side surface of said second half portion opposite said second pad and aligned or adjacent therewith; and
said secondary mount configured for engagement of a supplemental reservoir of a said anti pathogenic agent.

15. The securement device of claim 13 additionally comprising:
said secondary mount formed on said side surface of said second half portion opposite said second pad and aligned or adjacent therewith also configured for an operative engagement with a supplemental reservoir of a said anti pathogenic agent.

16. The securement device of claim 14 additionally comprising:
said secondary mount formed on said side surface of said second half portion opposite said second pad and aligned or adjacent therewith also configured for an operative engagement with a supplemental reservoir of a said anti pathogenic agent.
